(19) United States
(12) Patent Application Publication
(10) Pub. No.: US 2025/0321334 A1
(43) Pub. Date: Oct. 16, 2025

(54) Geological Disaster Monitoring Method, Device, Medium and Product

(71) Applicant: Chengdu University of Technology, Chengdu City (CN)

(72) Inventors: Lina HAO, Chengdu City (CN); Weile LI, Chengdu City (CN); Guang ZHENG, Chengdu City (CN); Qiang XU, Chengdu City (CN)

(73) Assignee: Chengdu University of Technology, Chengdu City, SC (CN)

(21) Appl. No.: 18/813,109

(22) Filed: Aug. 23, 2024

(30) Foreign Application Priority Data
Apr. 16, 2024 (CN) 202410454118.5

(51) Int. Cl. G01S 13/90; G01N 33/24; G01S 13/88; G01V 20/00

(52) U.S. Cl. CPC G01S 13/9023; G01N 33/24; G01S 13/885; G01V 20/00

(57) ABSTRACT

A geological disasters monitoring method, device, medium and product are provided, which relates to the technical field of geological monitoring. The method includes determining microscopic deformation parameters of an area to be monitored according to remote sensing observation data corresponding to a slope body of the area to be monitored, determining macroscopic deformation parameters of the area to be monitored according to optical remote sensing data and terrain data of the area to be monitored, and determining the landslide remote sensing geomechanical deformation type in the area to be monitored according to material composition, movement mode, slope structure, the microscopic deformation parameters and the macroscopic deformation parameters of the area to be monitored. The present disclosure improves the accuracy of monitoring geological disasters.

### CROSS-REFERENCE TO RELATED APPLICATION

This patent application claims the benefit and priority of Chinese Patent Application No. 2024104541185 filed with the China National Intellectual Property Administration on Apr. 16, 2024, the disclosure of which is incorporated by reference herein in its entirety as part of the present application.

### TECHNICAL FIELD

The present disclosure relates to the technical field of geological monitoring, in particular to method for monitoring geological disasters, a device thereof, a medium and a product.

### BACKGROUND

Large-scale geological disasters are often located at high altitudes with difficult transportation and few people. It is difficult to study their genetic model and deformation mechanism only by means of traditional field investigation and mass prediction and disaster prevention. It is necessary to study the long time sequence deformation process of a landslide with the help of modern high-precision earth observation technology (high-resolution optical remote sensing technology, Interferometric Synthetic Aperture Radar (InSAR), etc.), and to correlate their genetic model according to deformation features, which is helpful to the monitoring and early warning of a high-altitude remote landslide.

The macroscopic deformation of geological disasters is mainly manifested in the dynamic changes of topography and geomorphology, obvious cracks on a disaster body and a small-scale pre-sequence collapse. The above-mentioned obvious deformation is incoherent on SAR images, which cannot be dynamically monitored by the InSAR technology. However, the development of the macroscopic deformation is, to some extent, the external manifestation of microscopic deformation accumulation to some extent. Therefore, the tracking of the macroscopic deformation is the key to reveal a coupling process and a coupling mechanism of the macroscopic-microscopic deformations. At present, the development of optical remote sensing technology with a high spatial resolution and a high time-phase resolution provides an opportunity to track the macroscopic deformation features of geological disasters, which can realize the dynamic tracking and analysis of the features of disaster-pregnant background, topography and geomorphology, and obvious cracks, including the monitoring and extraction of boundaries of a geomorphic unit and the dynamic monitoring of cracks of a slope body.

The InSAR technology is a hot spot in the application of microscopic deformation detection and accurate measurement, and it is also a common technology in the detection, monitoring and early warning of hidden dangers of geological disasters. With the rapid development of the computer software and hardware technology, the InSAR technology is constantly innovating, and methods such as a Differential Interferometric Synthetic Aperture Radar (D-InSAR) technology, a Persistent Scatter InSAR (PSI) method, an Small Baselines Subset (SBAS) method and an Multiple Aperture InSAR (MAI) technology have emerged, which have made remarkable achievements in removing atmospheric effects and improving measurement accuracy. However, there are still many key problems to be solved urgently, such as uncertainty resulted from incoherence, an atmospheric delay and an orbit error, and insensitivity to the north-south deformation. In order to solve the problem that there is a large amount of phase unwrapping calculation in the InSAR algorithm and the D-InSAR cannot acquire a large-magnitude deformation, Michel et al. first proposed the Pixel Offset Tracking (POT) technology in 1999. This method can obtain better deformation information without unwrapping or being affected by image coherence, which has good application and reliability in deformation/displacement monitoring of earthquakes, glaciers and landslides. The POT technology includes a coherence tracking method and an intensity tracking method. The intensity tracking method can be used to register SAR images using the method of matching images with gray information with reference to optical images, and can also be used to carry out sub-pixel registration of multi-phase optical images. Therefore, the POT technology can be used for both deformation analysis of SAR images and horizontal deformation analysis of multi-phase optical images. Therefore, the optical image deformation analysis based on the POT technology can make up for the problem of insensitivity to the north-south deformation in the radar image deformation analysis based on the InSAR technology.

Optical remote sensing images can effectively identify areas with obvious deformation signs, but they are easily affected by cloud and fog weather and vegetation coverage. The optical images are not reflected clearly when the deformation signs are not obvious at the initial stage of deformation. The InSAR technology can effectively identify large areas that are slowly deforming, but the technology is easily restricted by an observation angle, vegetation coverage, water vapor and data processing technology. Therefore, the accuracy of monitoring geological disasters needs to be improved.

### SUMMARY

The present disclosure aims to provide a method of monitoring geological disasters, a device thereof, a medium and a product, which improves the accuracy of monitoring geological disasters.

In order to achieve the above objectives, the present disclosure provides the following scheme.

A method of monitoring geological disasters is provided, where the method includes:
determining microscopic deformation parameters of an area to be monitored according to remote sensing observation data corresponding to a slope body of the area to be monitored;
determining macroscopic deformation parameters of the area to be monitored according to optical remote sensing data and terrain data of the area to be monitored; and
determining the landslide remote sensing geomechanical deformation type in the area to be monitored according to material composition, movement mode, slope structure, the microscopic deformation parameters and the macroscopic deformation parameters of the area to be monitored.

In some embodiments, the determining microscopic deformation parameters of an area to be monitored according to remote sensing observation data corresponding to a slope body of the area to be monitored includes:
in a case that the slope body of the area to be monitored is an east-west slope body, using Interferometric Synthetic Aperture Radar (InSAR) technology to invert the time sequence microscopic deformation process of a landslide body according to the time sequence data of radar satellite images, and determining microscopic deformation data according to the time sequence microscopic deformation process, where the microscopic deformation data includes a microscopic deformation magnitude and a deformation area; and
in a case that the slope body of the area to be monitored is a north-south slope body, using POT technology to acquire the microscopic deformation data of the area to be monitored according to optical image time sequence data.

In some embodiments, the determining microscopic deformation data according to the time sequence microscopic deformation process includes:
extracting an average deformation velocity and an accumulated deformation amount from the time sequence microscopic deformation process;
determining an average deformation rate according to the deformation velocity and the accumulated deformation amount; and
determining the microscopic deformation magnitude according to the average deformation rate.

In some embodiments, when the average deformation rate is more than 100 mm/a, the microscopic deformation magnitude is a large-scale microscopic deformation, when the average deformation rate is more than 50 mm/a and less than or equal to 100 mm/a, the microscopic deformation magnitude is a medium-scale microscopic deformation, and when the average deformation rate is less than or equal to 50 mm/a, the microscopic deformation magnitude is a small-scale microscopic deformation.

In some embodiments, the determining macroscopic deformation parameters of the area to be monitored according to optical remote sensing data and terrain data of the area to be monitored includes:
extracting a local slump area in the area to be monitored by using a random forest classification method according to the optical remote sensing data;
extracting cracks and gullies in the area to be monitored by using an edge detection and random forest classification method according to the terrain data and the optical remote sensing data, where the terrain data include a multi-phase digital elevation model; and
determining macroscopic deformation magnitude in the area to be monitored according to an area ratio of the local slump area to the area to be monitored.

In some embodiments, when the area ratio is more than 25%, the macroscopic deformation magnitude is a large-scale macroscopic deformation, when the area ratio is less than or equal to 25% and more than 10%, the macroscopic deformation magnitude is a medium-scale macroscopic deformation, and when the area ratio is less than or equal to 10%, the macroscopic deformation magnitude is a small-scale macroscopic deformation.

In some embodiments, the landslide remote sensing geomechanical deformation type includes a soil thrust load caused landslide, a soil retrogressive landslide, a rock counter-tilt landslide, a block rock mass landslide, a rock flat thrust load caused landslide and a rock bedding landslide.

In the soil thrust load caused landslide, the material composition is soil, the movement mode is thrust load caused sliding, the slope structure is soil, the microscopic deformation parameter is a large-scale microscopic deformation in the middle and upper part of the landslide or a medium-scale microscopic deformation in the middle and upper part of the landslide, the macroscopic deformation parameter indicates that the local slump area is medium-scale or small-scale, and the macroscopic deformation parameters further indicates that the crack has a length of more than 10 meters.

In the soil retrogressive landslide, the material composition is soil, the movement mode is retrogressive sliding, the slope structure is soil, the microscopic deformation parameter is a large-scale microscopic deformation in lower part of the landslide or a medium-scale microscopic deformation in the lower part of the landslide, the macroscopic deformation parameter indicates that the local slump area is medium-scale or small-scale, and the macroscopic deformation parameter further indicates that the crack has a length of more than 10 meters, where the local slump area is located in the lower part of the landslide body.

In the rock counter-tilt landslide, the material composition is a rock, the movement mode is rotating sliding, the slope structure is a counter-tilt slope, the microscopic deformation parameter is a large-scale microscopic deformation in the lower part of the landslide or a medium-scale microscopic deformation in the lower part of the landslide, the macroscopic deformation parameter indicates that the local slump area is large-scale or medium-scale, and the macroscopic deformation parameters further indicates that there is a crack with a length of more than 10 meters and a width of more than 1 meter, where the local slump area is located in the middle part of the landslide body.

In the block rock mass landslide, the material composition is a rock, the movement mode is rotating sliding, the slope structure is a block slope, the microscopic deformation parameter is a large-scale microscopic deformation in the middle and upper part of the landslide or a medium-scale microscopic deformation in the middle and upper part of the landslide, the macroscopic deformation parameter indicates that the local slump area is large-scale or medium-scale, and the macroscopic deformation parameter further indicates that the crack has a length of more than 10 meters, where the local slump area is located in the lower part of the landslide body.

In the rock flat-thrust load caused landslide, the material composition is a rock, the movement mode is flat-thrust load caused sliding, the slope structure is nearly horizontal, the microscopic deformation parameter is none, and the macroscopic deformation parameter indicates that the crack has a width of more than 10 meters.

In the rock bedding landslide, the material composition is a rock, the movement mode is plane sliding, the slope structure is a bedding slope, the microscopic deformation parameter is none, the macroscopic deformation parameter indicates that the local slump area is small-scale, and the macroscopic deformation parameter further indicates that the crack has a length of more than 10 meters, where the local slump area is located in the lower part of the landslide body.

In another aspect, computer device is provided, including a memory, a processor and a computer program stored in the memory and operable on the processor, where the processor executes the computer program to implement the steps of the method of monitoring geological disasters.

In another aspect, a non-transitory computer-readable storage medium is provided, on which a computer program is stored, where the computer program, when executed by a processor, implements steps of the method of monitoring geological disasters.

In another aspect, a computer program product is provided, including a computer program, which when executed by a processor, implements steps of the geological disasters monitoring method.

According to the specific embodiments provided by the present disclosure, the present disclosure provides the following technical effects.

According to the present disclosure, the microscopic deformation parameters and the macroscopic deformation parameters of the area to be monitored are determined according to multi-source data, and the landslide remote sensing geomechanical deformation type in the area to be monitored is determined based on the microscopic deformation parameters and the macroscopic deformation parameters, so that the accuracy of monitoring and early warning of the landslide can be improved according to the landslide remote sensing geomechanical deformation type.

### BRIEF DESCRIPTION OF THE DRAWINGS

In order to explain the embodiments of the present disclosure or the technical schemes in the prior art more clearly, the drawings that need to be used in the embodiments will be briefly introduced hereinafter. Obviously, the drawings in the following description are only some embodiments of the present disclosure. For those skilled in the art, other drawings can be obtained according to these drawings without creative labor.

FIG. 1 is a schematic flow chart of a method for monitoring geological disasters according to an embodiment of the present disclosure;

FIG. 2 is a schematic diagram of a construction process of a landslide remote sensing geomechanical deformation model according to an embodiment of the present disclosure;

FIG. 3 is a schematic flow chart of acquiring a landslide microscopic deformation based on radar image data according to an embodiment of the present disclosure;

FIG. 4 is a schematic flow chart of acquiring a landslide deformation based on optical remote sensing data of Gaofen-2 satellite according to an embodiment of the present disclosure;

FIG. 5 is a picture of east-west deformation results of Baige landslide from 2015 to 2018 under different windows based on the POT technology according to an embodiment of the present disclosure;

FIG. 6 is a schematic flow chart of extracting a landslide macroscopic deformation by a random forest method according to an embodiment of the present disclosure;

FIG. 7 is a picture of a landslide boundary according to an embodiment of the present disclosure;

FIGS. 8A-8B are pictures of a local landslide slump sample according to an embodiment of the present disclosure;

FIG. 9 is a schematic diagram of a local landslide spectrum sample according to an embodiment of the present disclosure;

FIGS. 10A-10B are pictures of a vegetation sample according to an embodiment of the present disclosure;

FIGS. 11A-11B are pictures of a bare ground sample according to an embodiment of the present disclosure;

FIGS. 12A-12B are pictures of a waterbody sample according to an embodiment of the present disclosure;

FIGS. 13A-13B are pictures of a building sample according to an embodiment of the present disclosure;

FIG. 14 is a schematic diagram of a vegetation spectrum sample according to an embodiment of the present disclosure;

FIG. 15 is a schematic diagram of a bare ground spectrum sample according to an embodiment of the present disclosure;

FIG. 16 is a schematic diagram of a waterbody spectral sample according to an embodiment of the present disclosure;

FIGS. 17A-17B are schematic diagrams of a building spectrum sample according to an embodiment of the present disclosure;

FIG. 18 is a picture of an extraction result of local slump information of Baige landslide according to an embodiment of the present disclosure; and

FIG. 19 is an internal structure diagram of a computer device.

### DETAILED DESCRIPTION OF THE EMBODIMENTS

The technical schemes in the embodiments of the present disclosure will be clearly and completely described with reference to the drawings in the embodiments of the present disclosure hereinafter. Obviously, the described embodiments are only some embodiments of the present disclosure, rather than all of the embodiments. Based on the embodiment of the present disclosure, all other embodiments obtained by those skilled in the art without creative labor fall within the scope of protection of the present disclosure.

The present disclosure aims to provide a method for monitoring geological disasters, a device thereof, a medium and a product, which improves the accuracy of monitoring geological disasters.

In order to make the above objectives, features and advantages of the present disclosure more clear and understandable, the present disclosure will be explained in further detail with reference to the drawings and detailed description hereinafter.

### Embodiment 1

In this embodiment, with the support of sequences of high-resolution space-borne optical images and radar image data over a long time, the microscopic surface deformation parameters of landslide disasters are dynamically acquired by using time sequence Interferometric Synthetic Aperture Radar (InSAR) analysis technology and Pixel Offset Tracking (POT) analysis technology. Based on the landslide cause model, the sequences of high-resolution space-borne optical images and the Digital Terrain Model (DEM) data over a long time, the macroscopic deformation features of the landslide are dynamically acquired by using the deep learning information extraction algorithm and the geographic information space analysis and modeling technology, in which the macroscopic deformation features includes the change process of features such as disaster-pregnant background, disaster-pregnant micro-topography and obvious cracks. Based on the space-time matching model of microscopic-macroscopic deformation parameters of the landslide, the landslide remote sensing deformation model is constructed. Based on the existing landslide geomechanical models (19 models which are constructed from the slope material composition, the movement mode and the slope structure, and which are established by traditional means, specifically by field investigation information), the surface deformation trajectories of 19 traditional geomechanical models are analyzed and summarized from the perspective of remote sensing detection, the mapping relationship between the landslide remote sensing deformation model and the landslide geomechanical model is excavated, and the landslide remote sensing geomechanical deformation type is established. According to the present disclosure, the remote sensing information acquisition means is used to solve the mechanical deformation model of the remote high-altitude landslide that human beings cannot reach, which can acquire accurately the data in early warning and forecasting of the remote high-altitude landslide, in addition to saving manpower and material resources, and can assist the mechanical deformation model mechanism research of the remote high-altitude landslide.

Generally, landslides will undergo overall sliding only after a long period of slow deformation evolution. From the perspective of geomechanics, the concentrated stresses such as a tensile stress, a compressive stress, a shear stress produced and the like generated by each part of the landslide in different deformation and evolution processes of landslide bodies with different disaster-forming modes are different. For the different stress concentration areas, the corresponding areas inside the landslide produce the deformation corresponding to its mechanical properties. With the continuous accumulation of the deformation, cracks appear. The deformation development is slowly expanded, and then local slumping are developed. The geomorphic features of the slope where the landslide is located are changed accordingly. The slow and continuous microscopic deformation of the slope where the landslide is located, the continuous macro-expansion/increase of cracks and the occurring process of local slump features are related to the causes of the landslide and its geomechanical model, which are the key indicators for remote sensing, identifying and detecting of landslides. Therefore, the space-time deformation parameters of the landslide at different scales in different deformation processes can be obtained by using various remote sensing observation means. The landslide remote sensing geomechanical deformation type can be constructed based on the combination mode of space-time deformation parameters and their corresponding relationship with the landslide geomechanical evolution process, thus solving the problems of difficulty in acquiring deformation parameters of high-altitude remote landslides and studying geological models.

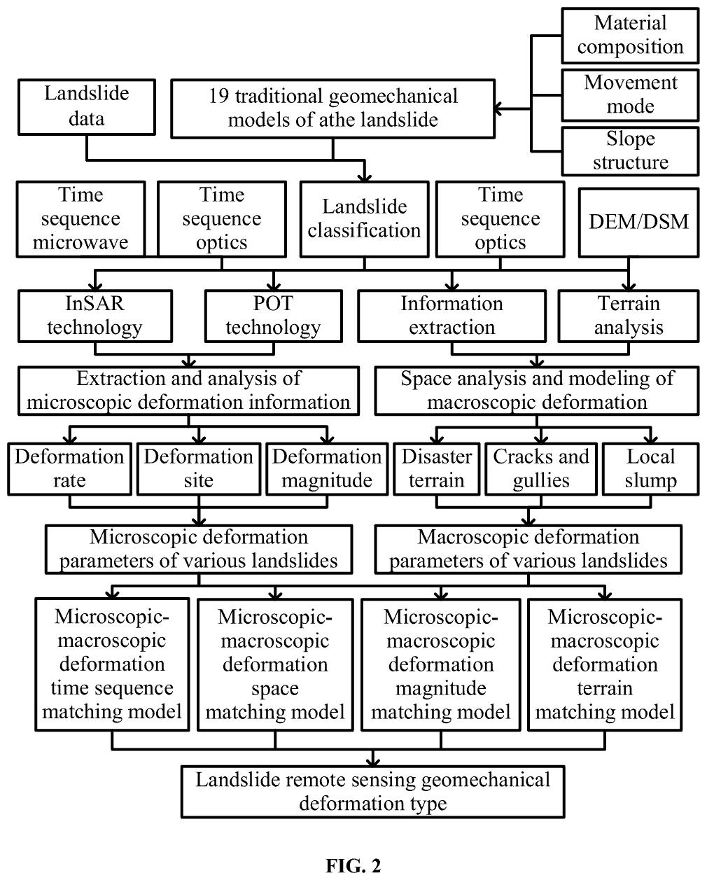

The technical idea of this embodiment is as follows: firstly, based on the existing 19 landslide geomechanical models, the existing landslide data is classified; secondly, based on microwave remote sensing data and optical remote sensing data, the microscopic deformation features of various landslides are acquired by using time sequence Interferometric Synthetic Aperture Radar (InSAR) analysis technology and Pixel Offset Tracking (POT) technology. At the same time, based on optical remote sensing data and terrain data, the macroscopic deformation information of various landslides is acquired by using various information automatic extraction and analysis methods. The macroscopic deformation features of different landslide types are revealed by using geographic information space analysis and modeling. Thereafter, based on the space-time matching mode of microscopic deformation parameters and macroscopic deformation parameters of different types of landslides, the space matching mode of microscopic deformation parameters and the macro-deformation parameters of various types of landslides is analyzed from the perspective of surface deformation signs in the movement process of various types of landslides, and the landslide remote sensing geomechanical deformation type is constructed to solve the geomechanical deformation model problem of remote high-altitude landslides. The specific technical route is shown in FIG. 2.

On the basis of the slope deformation destruction model, based on the key factors that control and influence the cause of landslides, starting with the slope movement mode and the material composition (a rock and soil), the slump disasters in western mountainous areas are divided into 19 genetic models through the analysis of landslide formation conditions and deformation and destruction basic laws, that is:

Dumping including (1) Block dumping, (2) Shallow dumping (rocks and soil), (3) Compression-dumping and (4) Deep dumping.

Sliding including (a), rotating sliding: (5) creep-cracking (soil), (6) creep-cracking-shearing, (7) compression-cracking-shearing, (8) collapse-cracking-shearing, (9) sliding-shearing; (b), plane sliding: (10) sliding-cracking (soil), (11) bedding sliding-cracking, (12) rotating sliding-cracking, (13) wedge sliding, (14) flat-thrust load caused sliding, (15) apparent dumping sliding-shearing, (16) plastic flow-cracking; (c), irregular sliding: (17) stepwise sliding, (18) sliding-supporting arch-shearing (soil), (19) sliding-bending-shearing. The scheme takes into account the slope movement mode, the material composition and he key disaster-causing factors, but the classification is too fine to identify each type of landslides from the perspective of remote sensing detection.

According to the existing 19 traditional geomechanical models, all the collected landslide data are classified, and each type of landslides will be used for the next calculation and statistical analysis of microscopic deformation parameters and macroscopic deformation parameters, respectively.

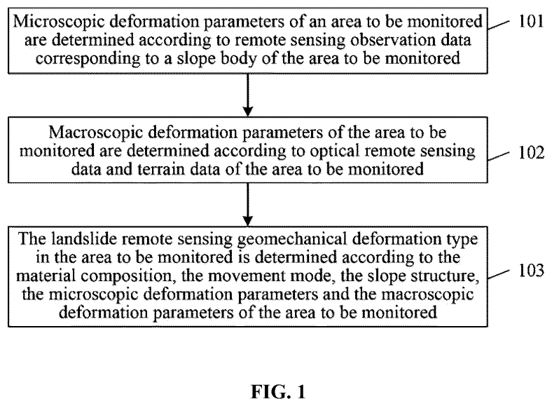

As shown in FIG. 1, a method for monitoring geological disasters in this embodiment includes the following steps 101-103.

In step 101, microscopic deformation parameters of an area to be monitored are determined according to remote sensing observation data corresponding to a slope body of the area to be monitored.

In step 102, macroscopic deformation parameters of the area to be monitored are determined according to optical remote sensing data and terrain data of the area to be monitored.

In step 103, the landslide remote sensing geomechanical deformation type in the area to be monitored is determined according to the material composition, the movement mode, the slope structure, the microscopic deformation parameters and the macroscopic deformation parameters of the area to be monitored.

Step 101 specifically includes:
when the slope body of the area to be monitored is an east-west slope body, using InSAR technology to invert the time sequence microscopic deformation process of a landslide body according to the time sequence data of radar satellite images, and
determining microscopic deformation data according to the time sequence microscopic deformation process, where the microscopic deformation data includes a microscopic deformation magnitude and a deformation area.

Determining microscopic deformation data according to the time sequence microscopic deformation process specifically includes:
extracting an average deformation velocity and an accumulated deformation amount from the time sequence microscopic deformation process; and
determining the microscopic deformation magnitude according to the average deformation rate.

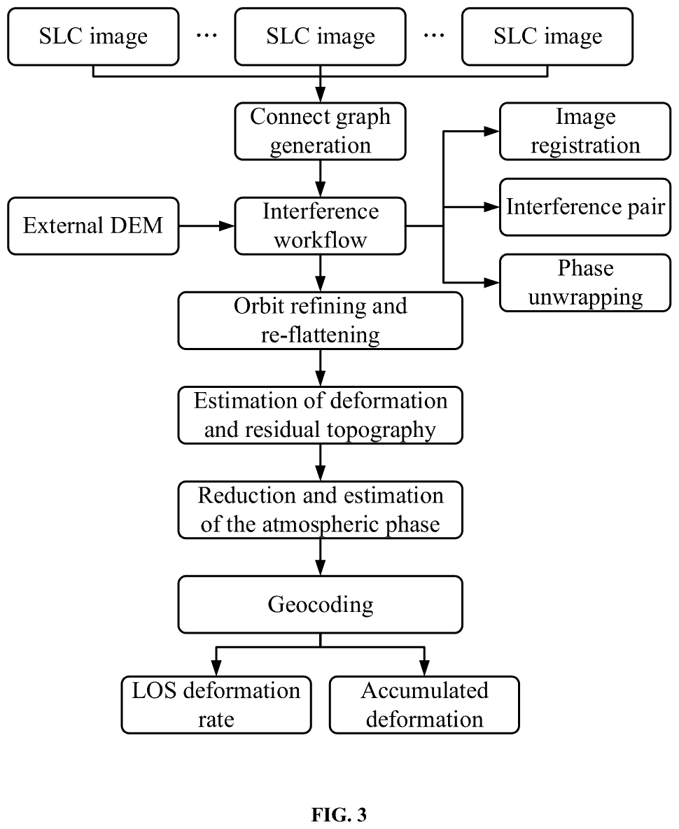

The process of acquiring microscopic deformation parameters of the landslide by using the InSAR technology is shown in FIG. 3, which specifically includes the following steps.
1) In step of image acquisition, long time sequence radar satellite remote sensing images and Digital Terrain Model (DEM) data are acquired.
2) In step of generating interference pairs, according to the time sequence t₀, t₁, . . . , tₙ, N+1 Single Look Complex (SLC) images are obtained. One of the images is selected as the main image to register with other images, and an appropriate space-time baseline constraint threshold is selected to generate interference pairs. N is the number of images, and tₙ is the n-th SLC image.
3) In step of unwrapping, the orbit information and the external DEM data are used, the interference pairs are differentially processed one by one, the flat effect and the terrain effect are removed to obtain multi-view differential interferograms, and the phase unwrapping is completed by using the Minimum Cost Flow (MCF) method. The orbit information includes instrument parameters, calibration parameters, orbit parameters, etc. In the present disclosure, the orbit information specifically refers to precise orbit determination ephemeris data.
4) In step of optimization, after removing the interference pairs containing a phase error and a low coherence, the stable Ground Control Points (GCPs) are selected for optimization. The residual constant phase and the phase ramp after unwrapping are removed by optimizing the re-flattening.
5) In step of first inversion, the linear model is used to estimate the deformation rate and the residual terrain, and the second unwrapping is carried out to optimize the input interferogram.
6) In step of second inversion, based on the existing deformation rate, atmospheric filtering is carried out to estimate and remove the atmospheric phase. Finally, the final deformation rate is obtained by a Least Square (LS) or Singular Value Decomposition (SVD) method, and the displacement in time sequence is calculated.
7) In step of geocoding, before geocoding, the deformation results are in the slant-range coordinates, and after geocoding, the results are output as geographical coordinates, so as to obtain the deformation rate and the deformation magnitude in the line-of-sight direction. According to the deformation rate and the accumulated deformation amount, the landslide microscopic deformation is classified. When the average deformation rate is more than 100 mm/a, the deformation is a large-scale microscopic deformation, when the average deformation rate is more than 50 mm/a and less than or equal to 100 mm/a, the deformation is a medium-scale microscopic deformation, and when the average deformation rate is less than or equal to 50 mm/a, the deformation is a small-scale microscopic deformation.
8) In step of Microscopic deformation site: the microscopic deformation site of the landslide is obtained by superposition of the deformation data and the space data.

Because of the imaging mechanism of radar satellite, the method is only sensitive to the east-west slope body. However, for the north-south slope body, the method of inverting the landslide body by using the InSAR technology is powerless in the aspect of extracting the microscopic deformation.

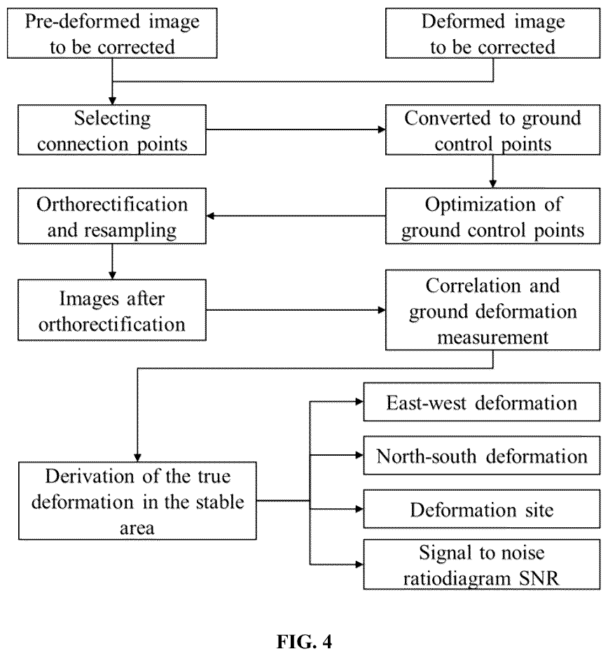

When the slope body of the area to be monitored is a north-south slope body, POT technology is used to acquire the microscopic deformation data of the area to be monitored according to optical image time sequence data. The POT pixel offset technology is used to dynamically track its deformation process, and the quantitative data such as the deformation magnitude, the deformation rate and the deformation area of the landslide is acquired. Based on the high-resolution optical remote sensing image (taking Gaofen-2 satellite as an example), the process of acquiring the landslide deformation by the using POT technology is shown in FIG. 4, which specifically includes the following steps.
1) In step of image selection, multi-period and multi-temporal remote sensing images of the same sensor (same satellite remote sensing data) are selected to ensure that the optical remote sensing images of the same sensor (such as Gaofen-2 satellite) within the research object satisfy 100% of overlap.
2) In step of image preprocessing, remote sensing images (optical images) are preprocessed, including radiometric calibration, atmospheric correction and image registration.
3) In step of image enhancement, the preprocessed remote sensing images are enhanced by principal component transformation. The first principal component feature band with the changed principal component is extracted for deformation detection, and the band information accounts for about 85% of the total information of the remote sensing images.
4) In step of deformation detection, the plug-in of Co-registration of Optically Sensed Images And Correlation (COSI-Corr) is used to debug and calculate parameters such as the window size (from 32-8 window to 512-512 window in FIG. 5). Based on the Fourier algorithm, the surface deformation data between images after principal component transformation in different phases are calculated, as shown in FIG. 5 (taking Baige landslide from 2015 to 2018 as an example). According to the surface deformation data, the landslide deformation is classified, the deformation below 10 meters is the medium-scale deformation, and the deformation above 10 meters is the large-scale deformation.

Step 102 specifically includes:
extracting a local slump area in the area to be monitored by using a random forest classification method according to optical remote sensing time sequence data;
using an edge detection and random forest classification method to extract cracks and gullies in the area to be monitored according to the terrain data and the optical remote sensing time sequence data, where the terrain data include a multi-phase digital elevation model; and
determining macroscopic deformation magnitude in the area to be monitored according to the area ratio of the local slump area to the area to be monitored.

When the area ratio is more than 25%, the deformation is a large-scale macroscopic deformation, when the area ratio is less than or equal to 25% and more than 10%, the deformation is a medium-scale macroscopic deformation, and when the area ratio is less than or equal to 10%, the deformation is a small-scale macroscopic deformation.

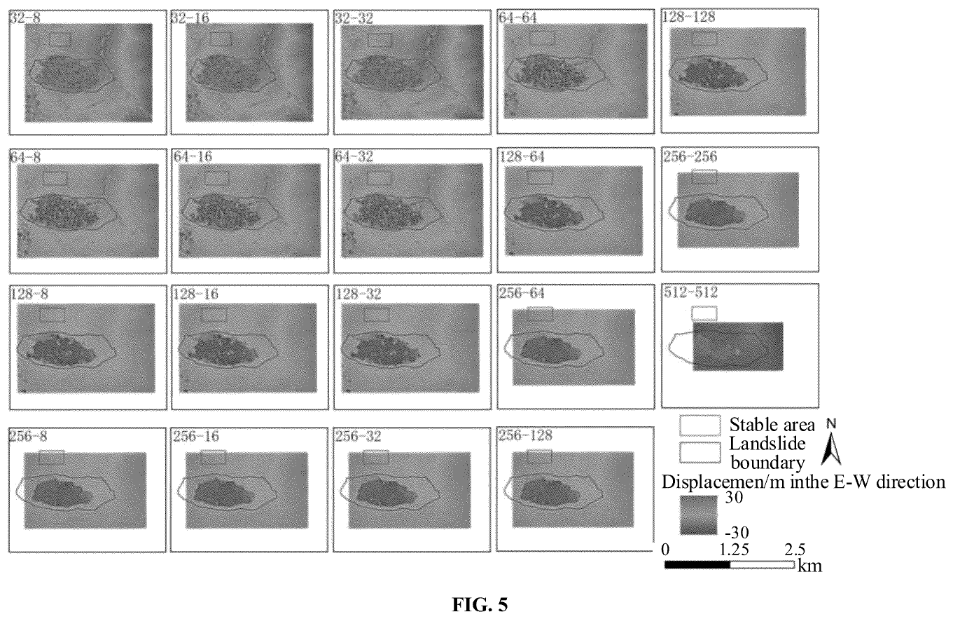

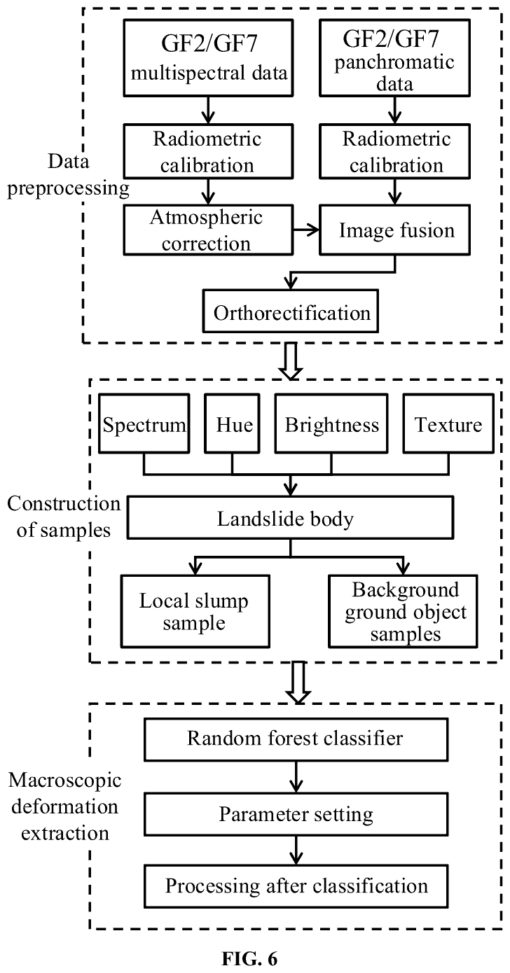

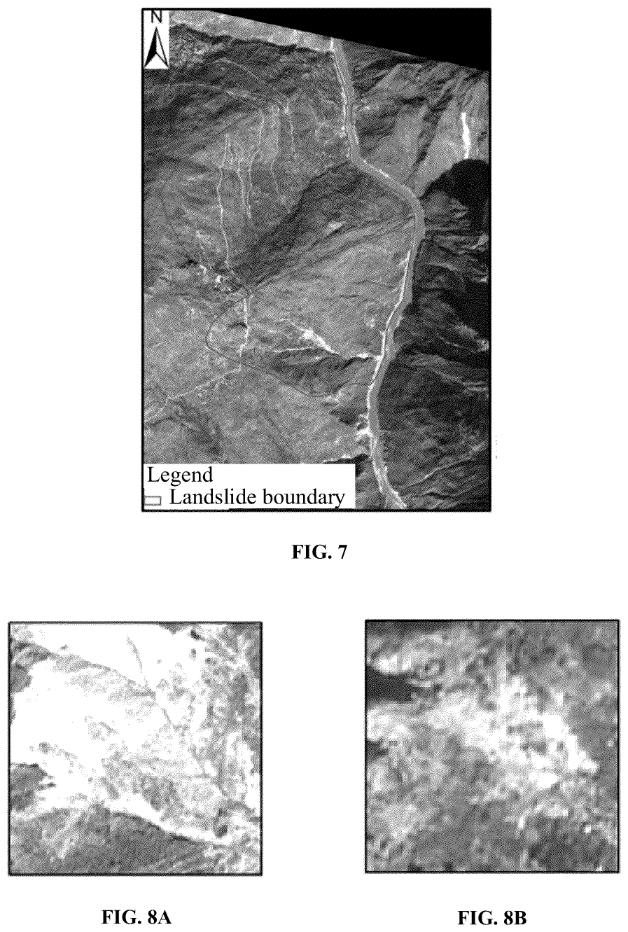

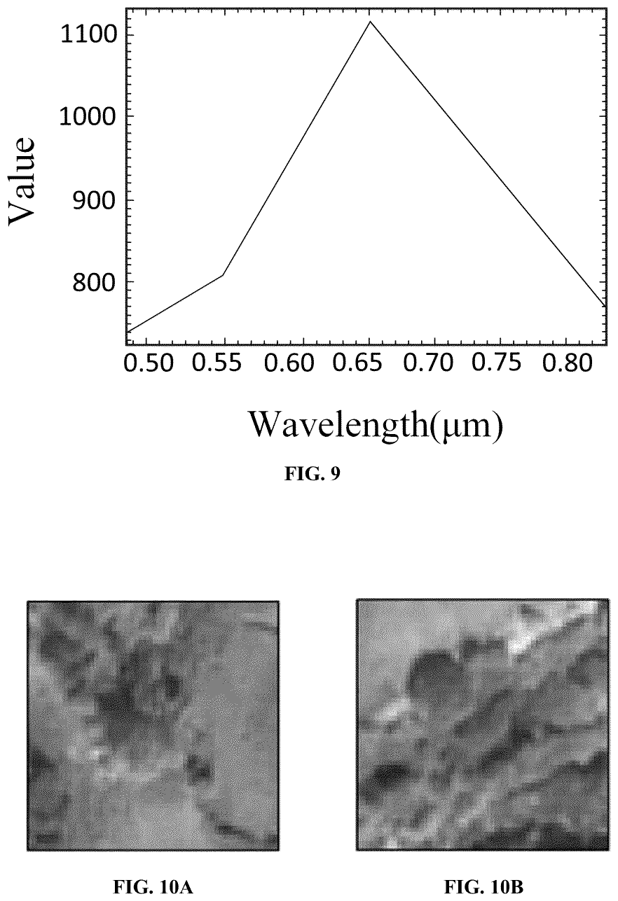

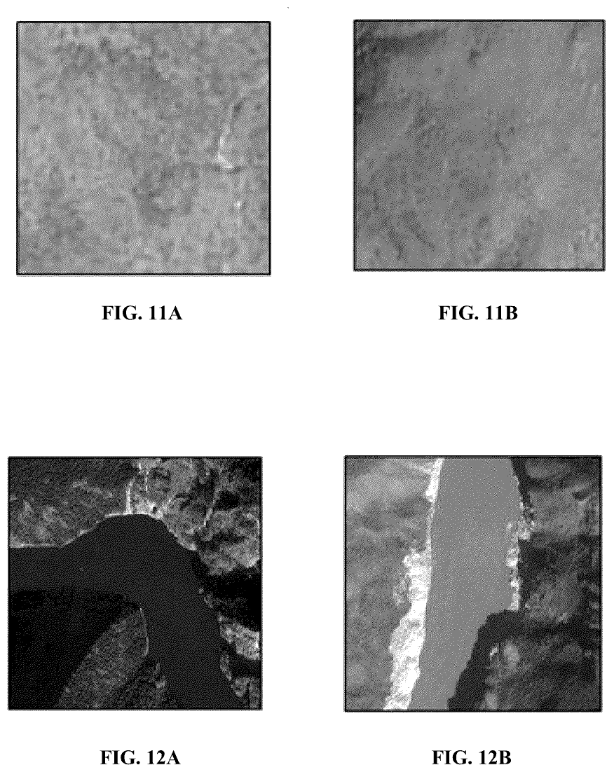

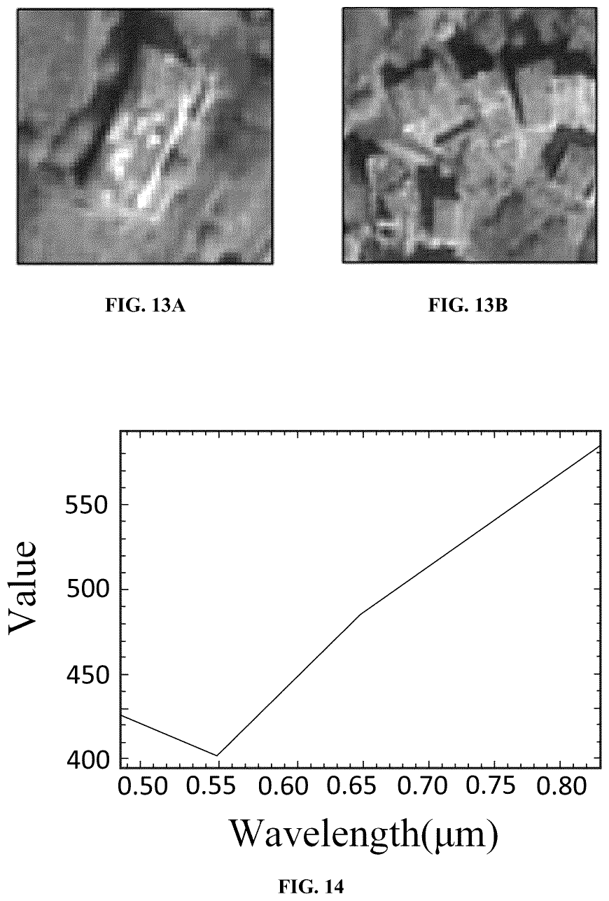

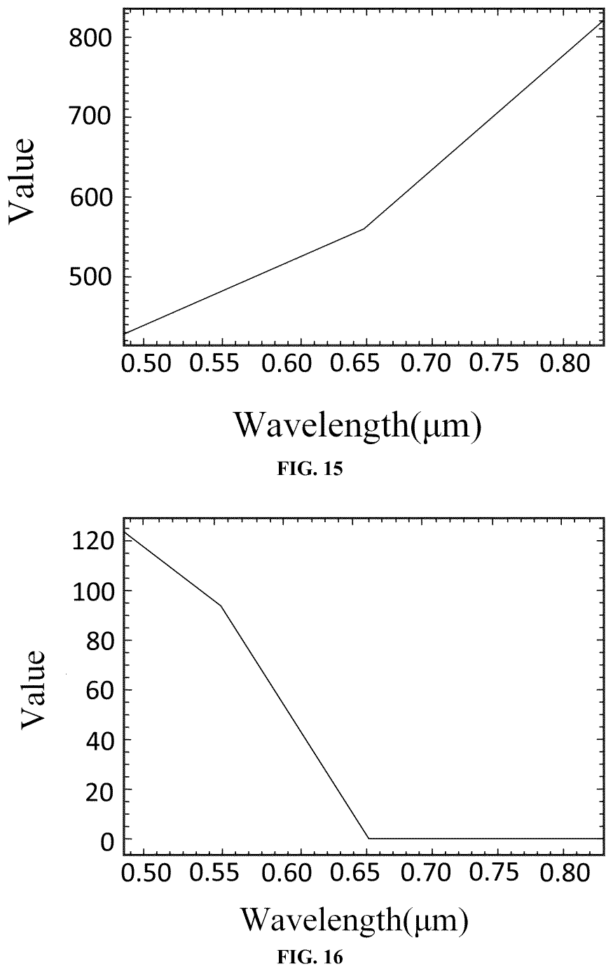

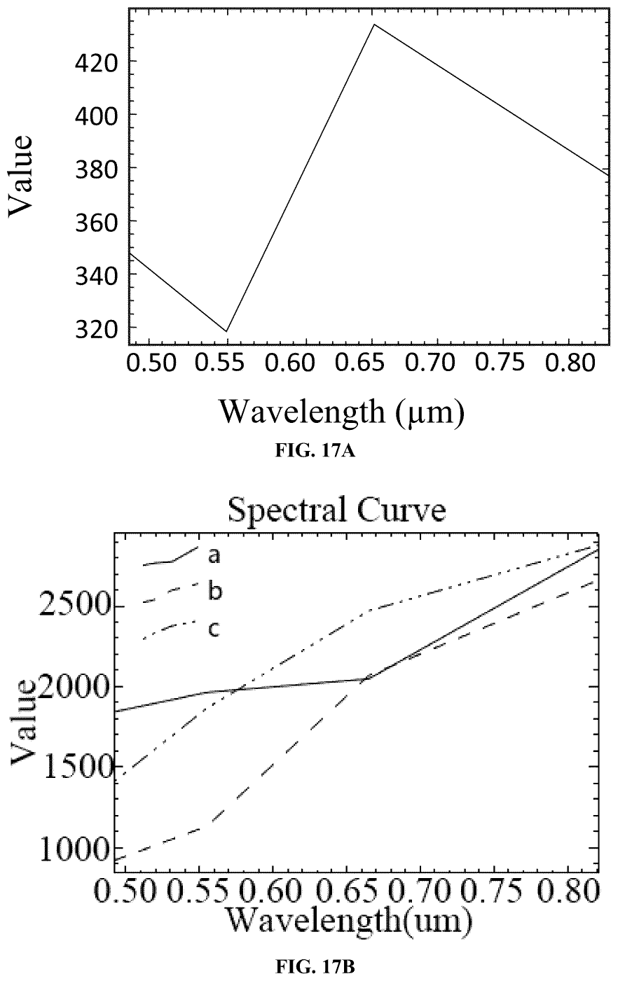

In order to acquire the macroscopic deformation parameters, based on long time sequence high-resolution optical remote sensing images, the macroscopic deformation information of the landslide body, that is, local slump information, is automatically extracted by using the random forest classification method, as shown in FIG. 6.
1) In step of data preprocessing, high-resolution optical remote sensing images (such as Gaofen-2 satellite and Gaofen-7 satellite) are subjected to data preprocessing such as radiation correction, atmospheric correction, image fusion, orthorectification and image enhancement.
2) In step of sample construction, according to the image feature of the remote sensing image such as hue, brightness, texture and spectrum, and the image feature of the local slump such as shape, hue, texture and spectrum, the local slump identification sign is established. Based on the established local slump sign, local slumps and landslides of the landslide body in the remote sensing images are selected to establish slump samples. At the same time, ground objects such as roads, bare lands, buildings, vegetation, water bodies in the background area are selected to construct non-slump samples, as shown in FIG. 7 to FIG. 17B.

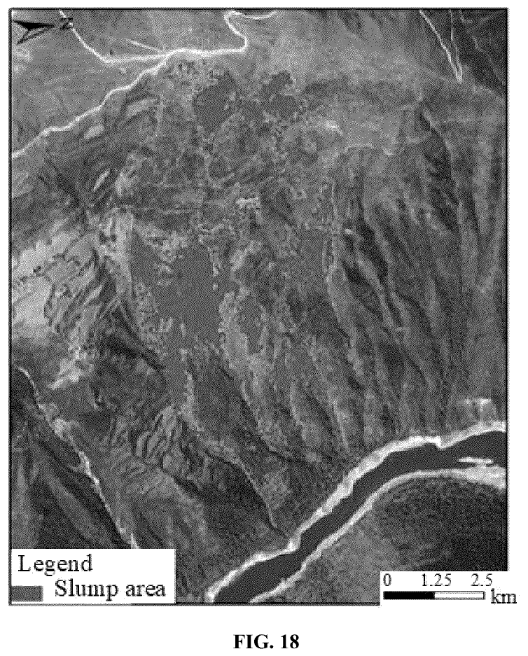

For extracting macro local slump information, the constructed local slump landslide samples and background ground object samples are imported. The classification parameters of the random forest are set to extract information (taking Baige landslide as an example), such as the local slump information extraction results of Baige landslide as shown in FIG. 18.
3) Based on the multi-period digital elevation model and the long time sequence high-resolution optical images, the edge detection and random forest classification method is used to automatically extract the information of cracks and gullies of the slump body.
4) The macroscopic deformation features of the slope where the landslide body is located are counted by using the space analysis method, including the macroscopic deformation site, deformation classification, etc. Macroscopic deformation classification is carried out based on the area ratio of the local slump area to the overall landslide area.

The landslide remote sensing geomechanical deformation type is determined.
1) The microscopic and macroscopic deformation parameters of landslide remote sensing are calculated and analyzed, in which the macroscopic deformation parameters and the microscopic deformation parameters of each type of a large number of landslides are calculated by Step 101 and Step 102, respectively.
2) The microscopic-macroscopic space-time coupling form of landslide remote sensing is analyzed and summarized.

The space-time matching model of microscopic deformation parameters and macroscopic deformation parameters of each type of landslides is analyzed, in which the space-time matching mode includes the features such as the deformation magnitude, the deformation order and the space distribution. The similarities and differences of the remote sensing deformation features of 19 traditional geomechanical models of landslides are excavated, the geological models and the remote sensing deformation features are integrated, and the landslide types with similar features are summarized and merged. The landslide remote sensing deformation models are shown in Table 1.

TABLE 1
landslide remote sensing deformation model

| landslide | microscopic deformation | | | | | |
|---|---|---|---|---|---|---|
| remote | parameter | | macroscopic deformation parameter | | | |
| sensing deformation | deformation | | local slump | | | |
| model | scale | deformation site | scale | site | cracks | gullies |
| microscopic- | large/ | the | large/ | the | development/ | development/ |
| macroscopic - | medium- | upper/middle/ | medium- | upper/middle/ | no development | no development |
| type | scale | lower part of the | scale | lower part of the | | |
| | | landslide | | landslide | | |
| Significant | medium/ | the | small- | the | none | no development |
| microscopic | large- | upper/middle/ | scale/ | upper/middle/ | | |
| deformation- | scale | lower part of the | none | lower part of the | | |
| type | | landslide | | landslide | | |
| microscopic- | small-scale | the | large/ | the | development | development/ |
| macroscopic - | | upper/middle/ | medium- | upper/middle/ | | no development |
| type | | lower part of the | scale | lower part of the | | |
| | | landslide | | landslide | | |
| Significant | small- | the | large/ | the | Significant | development/ |
| macroscopic | scale/ | upper/middle/ | medium- | upper/middle/ | | no development |
| deformation- | none | lower part of the | scale | lower part of the | | |
| type | | landslide | | landslide | | |

3) The landslide remote sensing geomechanical deformation type is constructed.

The analysis and summary results of the microscopic-macroscopic space-time coupling form of landslide remote sensing are analyzed and summarized based on the microscopic-macroscopic deformation coupling space-time matching of landslide remote sensing, the geomechanical deformation process and the deformation destruction model, and 6 types of landslide remote sensing geomechanical deformations are constructed.

According to the remote sensing geomechanical deformation type proposed by the present disclosure, starting from the limit and the detection indicator of remote sensing detection, starting from the material composition and the deformation destruction process of the slope, and integrating the detection indicator of remote sensing and the surface deformation signs of slope deformation and destruction, two classes of 6 landslide remote sensing geomechanical deformation types are finally proposed, that is:
1) Soil landslides includes (1) a thrust load caused landslide, and (2) a retrogressive landslide.
2) Rock landslides includes (3) a flat-thrust load caused landslide, (4) a bedding landslide, (5) a counter-tilt landslide, and (5) a block rock mass landslide.

The remote sensing deformation model of each landslide and the corresponding microscopic-macroscopic deformation parameters are shown in Table 2.

TABLE 2
landslide remote sensing geomechanical type and the remote sensing detection features thereof.

| | material | movement | slope | microscopic | macroscopic |
|---|---|---|---|---|---|
| type | composition | mode | structure | deformation parameter | deformation parameter |
| soil thrust | soil | thrust load | soil | The microscopic | the local slump area is |
| load caused | | caused sliding | | deformation in the | medium-scale or |
| landslide | | | | middle and upper part | small-scale, and the |
| | | | | of the landslide is large- | crack at the trailing |
| | | | | scale or medium-scale | edge is significant |
| soil | soil | retrogressive | soil | The microscopic | the local slump area in |
| retrogressive | | sliding | | deformation in the | the lower part of the |
| landslide | | | | lower part of the | landslide body is |
| | | | | landslide is large-scale | medium-scale or |
| | | | | or medium-scale | small-scale and the |
| | | | | | crack at the trailing |
| | | | | | edge is significant |
| rock counter- | rock | rotating sliding | counter-tilt | The microscopic | the local slump area in |
| tilt landslide | | | slope | deformation in the | the middle part of the |
| | | | | lower part of the | landslide body is |
| | | | | landslide is large-scale | large-scale or medium- |
| | | | | or medium-scale | scale and the crack |
| | | | | | develops at the trailing |
| | | | | | edge |
| block rock | rock | rotating sliding | block slope | The microscopic | the local slump area in |
| mass | | | | deformation in the | the lower part of the |
| landslide | | | | middle and upper part | landslide body is |
| | | | | of the landslide is large- | large-scale or medium- |
| | | | | scale or medium-scale | scale and the crack at |
| | | | | | the trailing edge is |
| | | | | | significant |
| rock flat- | rock | flat-thrust load | nearly | None | the wide crack at the |
| thrust load | | caused sliding | horizontal | | trailing edge is |
| caused | | | | | significant |
| landslide | | | | | |
| rock bedding | rock | plane sliding | bedding | None | the local slump area in |
| landslide | | | slope | | the lower part of the |
| | | | | | landslide body is |
| | | | | | small-scale, and the |
| | | | | | landslide boundary |
| | | | | | and the crack are |
| | | | | | significant |

The landslide remote sensing geomechanical deformation type in Step 103 includes a soil thrust load caused landslide, a soil retrogressive landslide, a rock counter-tilt landslide, a block rock mass landslide, a rock flat thrust load caused landslide and a rock bedding landslide.

In the specific application, the material composition and the slope structure can be judged step by step with the help of geological maps and other data. The movement mode can be judged according to the macroscopic-microscopic combination mode, and finally the remote sensing geomechanical type can be determined.

In the soil thrust load caused landslide, the material composition is soil, the movement mode is thrust load caused sliding, the slope structure is soil, the microscopic deformation parameter is a large-scale microscopic deformation in the middle and upper part of the landslide or a medium-scale microscopic deformation in the middle and upper part of the landslide, the macroscopic deformation parameter indicates that the local slump area is medium-scale or small-scale, and the crack at the trailing edge is significant.

In the soil retrogressive landslide, the material composition is soil, the movement mode is retrogressive sliding, the slope structure is soil, the microscopic deformation parameter is a large-scale microscopic deformation in the lower part of the landslide or a medium-scale microscopic deformation in the lower part of the landslide, the macroscopic deformation parameter indicates that the local slump area is medium-scale or small-scale and the crack at the trailing edge is significant, and the significant crack at the trailing edge indicates that the crack has a length of more than 10 meters, where the local slump area is located in the lower part of the landslide body.

In the rock counter-tilt landslide, the material composition is a rock, the movement mode is rotating sliding, the slope structure is a counter-tilt slope, the microscopic deformation parameter is a large-scale microscopic deformation in the lower part of the landslide or a medium-scale microscopic deformation in the lower part of the landslide, the macroscopic deformation parameter indicates that the local slump area is large-scale or medium-scale and the crack develops at the trailing edge. The crack development at the trailing edge specifically indicates that there is a crack with a length of more than 10 meters and a width of more than 1 meter, where the local slump area is located in the middle part of the landslide body.

In the block rock mass landslide, the material composition is a rock, the movement mode is rotating sliding, the slope structure is a block slope, the microscopic deformation parameter is a large-scale microscopic deformation in the middle and upper part of the landslide or a medium-scale microscopic deformation in the middle and upper part of the landslide, the macroscopic deformation parameter indicates that the local slump area is large-scale or medium-scale and the crack at the trailing edge is significant, where the local slump area is located in the lower part of the landslide body.

In the rock flat-thrust load caused landslide, the material composition is a rock, the movement mode is flat-thrust load caused sliding, the slope structure is nearly horizontal, the microscopic deformation parameter is none, and the macroscopic deformation parameter indicates that the wide crack at the trailing edge is significant, and the significant wide crack at the trailing edge indicates that the crack has a width of more than 10 meters.

In the rock bedding landslide, the material composition is a rock, the movement mode is plane sliding, the slope structure is a bedding slope, the microscopic deformation parameter is none, the macroscopic deformation parameter indicates that the local slump area is small-scale, and the landslide boundary and the crack are significant, where the local slump area is located in the lower part of the landslide body.

Remote sensing images can be used to judge the trailing edge and the leading edge of the landslide. The part near the trailing edge is the upper part or middle-upper part, and the part near the leading edge is the lower part or the middle-lower part.

At present, at home and abroad, the research focuses on the method of extracting thematic information such as landslide morphology, appearance, cover change and surface deformation by the technology such as high-resolution optical remote sensing technology and space-borne Interferometric Synthetic Aperture Radar (InSAR) technology. However, the relationship between the endogenous structure, the external performance and the deformation mechanism of disasters and comprehensive remote sensing image morphology and deformation observation has not yet been established. The evolution law of geological disasters (landslides) from the quantitative change accumulation stage of the microscopic deformation to the qualitative change stage of instable sliding is studied, that is, a microscopic-macroscopic deformation coupling mode. The construction of remote sensing deformation parameters and process of geomechanics is one of the key problems to be solved urgently in the accurate identification of geological disasters through comprehensive remote sensing, and it is also a problem to be solved urgently in the remote high-altitude landslide research. The comprehensive application of the source remote sensing technology of the present disclosure provides technical support for tracking the macroscopic-microscopic deformation process of geological disasters and quantifying the deformation data, which is beneficial to improving the accuracy of monitoring geological disasters.

### Embodiment 2

A non-transitory computer-readable storage medium is provided, on which a computer program is stored, where the computer program, when executed by a processor, implements the steps of the method of monitoring geological disasters in Embodiment 1.

### Embodiment 3

A computer program product is provided, including a computer program, where the computer program, when executed by a processor, implements the steps of the method of monitoring geological disasters in Embodiment 1.

### Embodiment 4

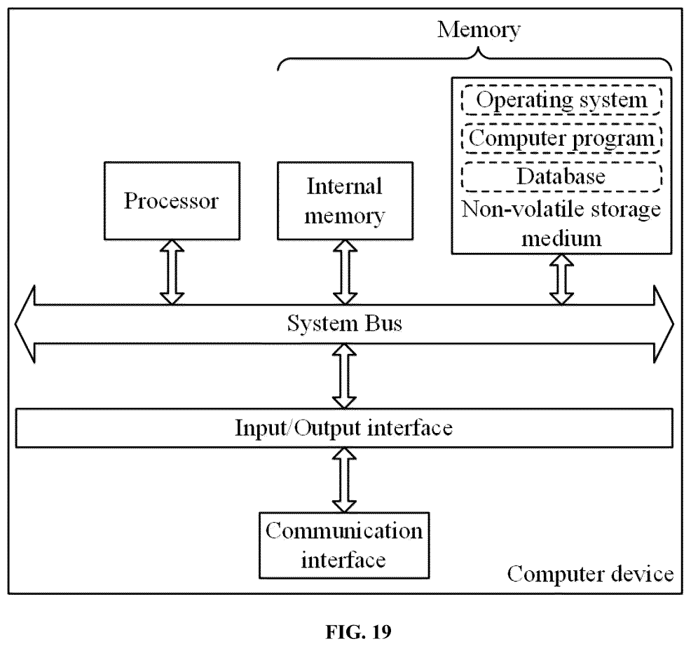

A computer device is provided, the internal structure of which can be shown in FIG. 19. The computer device includes a processor, a memory, an Input/Output interface (I/O for short) and a communication interface. The processor, the memory and the input/output interface are connected through the system bus, and the communication interface is connected to the system bus through the input/output interface. The processor of the computer device is configured to provide computing and control capabilities. The memory of the computer device includes a non-volatile storage medium and an internal memory. The non-volatile storage medium stores an operating system, a computer program and a database therein. The internal memory provides an environment for the operation of the operating system and the computer program in the non-volatile storage medium. The database of the computer device is configured to store pending transactions. The input/output interface of the computer device is configured to exchange information between the processor and the external device. The communication interface of the computer device is configured to communicate with the external terminal through network. The computer program, when executed by a processor, implements the method of monitoring geological disasters in Embodiment 1.

It should be noted that the object information (including but not limited to object device information, object personal information, etc.) and data (including but not limited to data for analysis, stored data, displayed data, etc.) involved in the present disclosure are all information and data authorized by the object or fully authorized by all parties, and the collection, use and processing of relevant data need to comply with relevant laws, regulations and standards of relevant countries and regions.

Those skilled in the art can understand that all or part of the processes in the method of implementing the above-mentioned embodiments can be completed by instructing related hardware through a computer program. The computer program can be stored in a non-volatile computer-readable storage medium, and when executed, the computer program can include the processes of the embodiments of the above-mentioned method. Any reference to the memory, the database or other media used in various embodiments provided by the present disclosure may include at least one of a non-volatile memory and a volatile memory. The non-volatile memory may include a Read-Only Memory (ROM), a magnetic tape, a floppy disk, a flash memory, an optical memory, a high-density embedded non-volatile memory, a Resistive Random Access Memory (ReRAM), a Magneto-Resistive Random Access Memory (MRAM), a Ferroelectric Random Access Memory (FRAM), a Phase Change Memory (PCM), a Graphene Memory, etc. The volatile memory may include a Random Access Memory (RAM) or an external cache memory. By way of illustration and not limitation, the RAM can be in various forms, such as a Static Random Access Memory (SRAM) or a Dynamic Random Access Memory (DRAM). The database involved in each embodiment provided by the present disclosure may include at least one of a relational database and a non-relational database. The non-relational database may include, but is not limited to, a distributed database based on a blockchain. The processor involved in various embodiments provided by the present disclosure may be, but is not limited to, a general processor, a central processing unit, a graphics processor, a digital signal processor, a programmable logic device, a data processing logic device based on quantum computing, etc.

The technical features of the above embodiments can be combined at will. In order to make the description concise, not all possible combinations of the technical features in the above embodiments are described. However, as long as there is no contradiction between the combinations of these technical features, they should be considered as the scope described in this specification.

In the present disclosure, specific examples are applied to illustrate the principle and implementation of the present disclosure, and the explanations of the above embodiments are only used to help understand the method and core ideas of the present disclosure. At the same time, according to the idea of the present disclosure, there will be some changes in the specific implementation and application scope for those skilled in the art. To sum up, the contents of the specification should not be construed as limiting the present disclosure.

What is claimed is:

1. A geological disasters monitoring method, comprising:
determining microscopic deformation parameters of an area to be monitored according to remote sensing observation data corresponding to a slope body of the area to be monitored;
determining macroscopic deformation parameters of the area to be monitored according to optical remote sensing data and terrain data of the area to be monitored; and
determining the landslide remote sensing geomechanical deformation type in the area to be monitored according to material composition, movement mode, slope structure, the microscopic deformation parameters and the macroscopic deformation parameters of the area to be monitored.

2. The geological disasters monitoring method according to claim 1, wherein the determining microscopic deformation parameters of an area to be monitored according to remote sensing observation data corresponding to a slope body of the area to be monitored comprises:
in a case that the slope body of the area to be monitored is an east-west slope body, using Interferometric Synthetic Aperture Radar (InSAR) technology to invert a time sequence microscopic deformation process of a landslide body according to time sequence data of radar satellite images, and determining microscopic deformation data according to the time sequence microscopic deformation process, wherein the microscopic deformation data comprises a microscopic deformation magnitude and a deformation area; and
in a case that the slope body of the area to be monitored is a north-south slope body, using Pixel offset tracking (POT) technology to acquire the microscopic deformation data of the area to be monitored according to optical image time sequence data.

3. The geological disasters monitoring method according to claim 2, wherein the determining microscopic deformation data according to the time sequence microscopic deformation process comprises:
extracting an average deformation velocity and an accumulated deformation amount from the time sequence microscopic deformation process;
determining an average deformation rate according to the deformation velocity and the accumulated deformation amount; and
determining the microscopic deformation magnitude according to the average deformation rate.

4. The geological disasters monitoring method according to claim 2, wherein when the average deformation rate is more than 100 mm/a, the microscopic deformation magnitude is a large-scale microscopic deformation, when the average deformation rate is more than 50 mm/a and less than or equal to 100 mm/a, the microscopic deformation magnitude is a medium-scale microscopic deformation, and when the average deformation rate is less than or equal to 50 mm/a, the microscopic deformation magnitude is a small-scale microscopic deformation.

5. The geological disasters monitoring method according to claim 1, wherein the determining macroscopic deformation parameters of the area to be monitored according to optical remote sensing data and terrain data of the area to be monitored comprises:
extracting a local slump area in the area to be monitored by using a random forest classification method according to the optical remote sensing data;
extracting cracks and gullies in the area to be monitored by using an edge detection and random forest classification method according to the terrain data and the optical remote sensing data, wherein the terrain data comprise a multi-phase digital elevation model; and
determining macroscopic deformation magnitude in the area to be monitored according to an area ratio of the local slump area to the area to be monitored.

6. The geological disasters monitoring method according to claim 5, wherein when the area ratio is more than 25%, the macroscopic deformation magnitude is a large-scale macroscopic deformation, when the area ratio is less than or equal to 25% and more than 10%, the macroscopic deformation magnitude is a medium-scale macroscopic deformation, and when the area ratio is less than or equal to 10%, the macroscopic deformation magnitude is a small-scale macroscopic deformation.

7. The geological disasters monitoring method according to claim 1, wherein the landslide remote sensing geomechanical deformation type comprises a soil thrust load caused landslide, a soil retrogressive landslide, a rock counter-tilt landslide, a block rock mass landslide, a rock flat thrust load caused landslide and a rock bedding landslide, and wherein:
in the soil thrust load caused landslide, the material composition is soil, the movement mode is thrust load caused sliding, the slope structure is soil, the microscopic deformation parameter is a large-scale microscopic deformation in middle and upper part of the landslide or a medium-scale microscopic deformation in the middle and upper part of the landslide, the macroscopic deformation parameter indicates that the local slump area is medium-scale or small-scale, and the macroscopic deformation parameters further indicates that the crack has a length of more than 10 meters;
in the soil retrogressive landslide, the material composition is soil, the movement mode is retrogressive sliding, the slope structure is soil, the microscopic deformation parameter is a large-scale microscopic deformation in lower part of the landslide or a medium-scale microscopic deformation in the lower part of the landslide, the macroscopic deformation parameter indicates that the local slump area is medium-scale or small-scale, and the macroscopic deformation parameter further indicates that the crack has a length of more than 10 meters, wherein the local slump area is located in the lower part of the landslide body;
in the rock counter-tilt landslide, the material composition is a rock, the movement mode is rotating sliding, the slope structure is a counter-tilt slope, the microscopic deformation parameter is a large-scale microscopic deformation in the lower part of the landslide or a medium-scale microscopic deformation in the lower part of the landslide, the macroscopic deformation parameter indicates that the local slump area is large-scale or medium-scale, and the macroscopic deformation parameters further indicates that there is a crack with a length of more than 10 meters and a width of more than 1 meter, wherein the local slump area is located in the middle part of the landslide body;
in the block rock mass landslide, the material composition is a rock, the movement mode is rotating sliding, the slope structure is a block slope, the microscopic deformation parameter is a large-scale microscopic deformation in the middle and upper part of the landslide or a medium-scale microscopic deformation in the middle and upper part of the landslide, the macroscopic deformation parameter indicates that the local slump area is large-scale or medium-scale, and the macroscopic deformation parameter further indicates that the crack has a length of more than 10 meters, wherein the local slump area is located in the lower part of the landslide body;
in the rock flat-thrust load caused landslide, the material composition is a rock, the movement mode is flat-thrust load caused sliding, the slope structure is nearly horizontal, the microscopic deformation parameter is none, and the macroscopic deformation parameter indicates that the crack has a width of more than 10 meters; and
in the rock bedding landslide, the material composition is a rock, the movement mode is plane sliding, the slope structure is a bedding slope, the microscopic deformation parameter is none, the macroscopic deformation parameter indicates that the local slump area is small-scale, and the macroscopic deformation parameter further indicates that the crack has a length of more than 10 meters, wherein the local slump area is located in the lower part of the landslide body.

8. A computer device comprising a memory, a processor and a computer program which is stored in the memory and operable on the processor, wherein the processor executes the computer program to implement steps of the geological disasters monitoring method according to claim 1.

9. The computer device according to claim 8, wherein the determining microscopic deformation parameters of an area to be monitored according to remote sensing observation data corresponding to a slope body of the area to be monitored comprises:
in a case that the slope body of the area to be monitored is an east-west slope body, using Interferometric Synthetic Aperture Radar (InSAR) technology to invert a time sequence microscopic deformation process of a landslide body according to time sequence data of radar satellite images, and determining microscopic deformation data according to the time sequence microscopic deformation process, wherein the microscopic deformation data comprises a microscopic deformation magnitude and a deformation area; and
in a case that the slope body of the area to be monitored is a north-south slope body, using Pixel offset tracking (POT) technology to acquire the microscopic deformation data of the area to be monitored according to optical image time sequence data.

10. The computer device according to claim 9, wherein the determining microscopic deformation data according to the time sequence microscopic deformation process comprises:
extracting an average deformation velocity and an accumulated deformation amount from the time sequence microscopic deformation process;
determining an average deformation rate according to the deformation velocity and the accumulated deformation amount; and
determining the microscopic deformation magnitude according to the average deformation rate.

11. The computer device according to claim 9, wherein when the average deformation rate is more than 100 mm/a, the microscopic deformation magnitude is a large-scale microscopic deformation, when the average deformation rate is more than 50 mm/a and less than or equal to 100 mm/a, the microscopic deformation magnitude is a medium-scale microscopic deformation, and when the average deformation rate is less than or equal to 50 mm/a, the microscopic deformation magnitude is a small-scale microscopic deformation.

12. The computer device according to claim 8, wherein the determining macroscopic deformation parameters of the area to be monitored according to optical remote sensing data and terrain data of the area to be monitored comprises:
extracting a local slump area in the area to be monitored by using a random forest classification method according to the optical remote sensing data;
extracting cracks and gullies in the area to be monitored by using an edge detection and random forest classification method according to the terrain data and the optical remote sensing data, wherein the terrain data comprise a multi-phase digital elevation model; and
determining macroscopic deformation magnitude in the area to be monitored according to an area ratio of the local slump area to the area to be monitored.

13. The computer device according to claim 12, wherein when the area ratio is more than 25%, the macroscopic deformation magnitude is a large-scale macroscopic deformation, when the area ratio is less than or equal to 25% and more than 10%, the macroscopic deformation magnitude is a medium-scale macroscopic deformation, and when the area ratio is less than or equal to 10%, the macroscopic deformation magnitude is a small-scale macroscopic deformation.

14. The computer device according to claim 8, wherein the landslide remote sensing geomechanical deformation type comprises a soil thrust load caused landslide, a soil retrogressive landslide, a rock counter-tilt landslide, a block rock mass landslide, a rock flat thrust load caused landslide and a rock bedding landslide, and wherein:
in the soil thrust load caused landslide, the material composition is soil, the movement mode is thrust load caused sliding, the slope structure is soil, the microscopic deformation parameter is a large-scale microscopic deformation in middle and upper part of the landslide or a medium-scale microscopic deformation in the middle and upper part of the landslide, the macroscopic deformation parameter indicates that the local slump area is medium-scale or small-scale, and the macroscopic deformation parameters further indicates that the crack has a length of more than 10 meters;
in the soil retrogressive landslide, the material composition is soil, the movement mode is retrogressive sliding, the slope structure is soil, the microscopic deformation parameter is a large-scale microscopic deformation in lower part of the landslide or a medium-scale microscopic deformation in the lower part of the landslide, the macroscopic deformation parameter indicates that the local slump area is medium-scale or small-scale, and the macroscopic deformation parameter further indicates that the crack has a length of more than 10 meters, wherein the local slump area is located in the lower part of the landslide body;
in the rock counter-tilt landslide, the material composition is a rock, the movement mode is rotating sliding, the slope structure is a counter-tilt slope, the microscopic deformation parameter is a large-scale microscopic deformation in the lower part of the landslide or a medium-scale microscopic deformation in the lower part of the landslide, the macroscopic deformation parameter indicates that the local slump area is large-scale or medium-scale, and the macroscopic deformation parameters further indicates that there is a crack with a length of more than 10 meters and a width of more than 1 meter, wherein the local slump area is located in the middle part of the landslide body;
in the block rock mass landslide, the material composition is a rock, the movement mode is rotating sliding, the slope structure is a block slope, the microscopic deformation parameter is a large-scale microscopic deformation in the middle and upper part of the landslide or a medium-scale microscopic deformation in the middle and upper part of the landslide, the macroscopic deformation parameter indicates that the local slump area is large-scale or medium-scale, and the macroscopic deformation parameter further indicates that the crack has a length of more than 10 meters, wherein the local slump area is located in the lower part of the landslide body;
in the rock flat-thrust load caused landslide, the material composition is a rock, the movement mode is flat-thrust load caused sliding, the slope structure is nearly horizontal, the microscopic deformation parameter is none, and the macroscopic deformation parameter indicates that the crack has a width of more than 10 meters; and
in the rock bedding landslide, the material composition is a rock, the movement mode is plane sliding, the slope structure is a bedding slope, the microscopic deformation parameter is none, the macroscopic deformation parameter indicates that the local slump area is small-scale, and the macroscopic deformation parameter further indicates that the crack has a length of more than 10 meters, wherein the local slump area is located in the lower part of the landslide body.

15. A non-transitory computer-readable storage medium on which a computer program is stored, wherein the computer program, when executed by a processor, implements steps of the geological disasters monitoring method according to claim 1.

16. The non-transitory computer-readable storage medium according to claim 15, wherein the determining microscopic deformation parameters of an area to be monitored according to remote sensing observation data corresponding to a slope body of the area to be monitored comprises:
in a case that the slope body of the area to be monitored is an east-west slope body, using Interferometric Synthetic Aperture Radar (InSAR) technology to invert a time sequence microscopic deformation process of a landslide body according to time sequence data of radar satellite images, and determining microscopic deformation data according to the time sequence microscopic deformation process, wherein the microscopic deformation data comprises a microscopic deformation magnitude and a deformation area; and
in a case that the slope body of the area to be monitored is a north-south slope body, using Pixel offset tracking (POT) technology to acquire the microscopic deformation data of the area to be monitored according to optical image time sequence data.

17. The non-transitory computer-readable storage medium according to claim 16, wherein the determining microscopic deformation data according to the time sequence microscopic deformation process comprises:
extracting an average deformation velocity and an accumulated deformation amount from the time sequence microscopic deformation process;
determining an average deformation rate according to the deformation velocity and the accumulated deformation amount; and
determining the microscopic deformation magnitude according to the average deformation rate.

18. The non-transitory computer-readable storage medium according to claim 16, wherein when the average deformation rate is more than 100 mm/a, the microscopic deformation magnitude is a large-scale microscopic deformation, when the average deformation rate is more than 50 mm/a and less than or equal to 100 mm/a, the microscopic deformation magnitude is a medium-scale microscopic deformation, and when the average deformation rate is less than or equal to 50 mm/a, the microscopic deformation magnitude is a small-scale microscopic deformation.

19. The non-transitory computer-readable storage medium according to claim 15, wherein the determining macroscopic deformation parameters of the area to be monitored according to optical remote sensing data and terrain data of the area to be monitored comprises:
extracting a local slump area in the area to be monitored by using a random forest classification method according to the optical remote sensing data;
extracting cracks and gullies in the area to be monitored by using an edge detection and random forest classification method according to the terrain data and the optical remote sensing data, wherein the terrain data comprise a multi-phase digital elevation model; and
determining macroscopic deformation magnitude in the area to be monitored according to an area ratio of the local slump area to the area to be monitored.

20. The non-transitory computer-readable storage medium according to claim 15, wherein the determining macroscopic deformation parameters of the area to be monitored according to optical remote sensing data and terrain data of the area to be monitored comprises:
extracting a local slump area in the area to be monitored by using a random forest classification method according to the optical remote sensing data;
extracting cracks and gullies in the area to be monitored by using an edge detection and random forest classification method according to the terrain data and the optical remote sensing data, wherein the terrain data comprise a multi-phase digital elevation model; and
determining macroscopic deformation magnitude in the area to be monitored according to an area ratio of the local slump area to the area to be monitored.
